(19) United States
(12) Patent Application Publication
(10) Pub. No.: US 2026/0086037 A1
(43) Pub. Date: Mar. 26, 2026

(54) UNIVERSAL PAPER-BASED GENOME DETECTION USING A UNIVERSAL RNA TOEHOLD SWITCH AND CRISPR

(71) Applicant: The Research Foundation for the State University of New York, Albany, NY (US)

(72) Inventors: Mehmet Yigit, Albany, NY (US); Mahera Kachwala, Albany, NY (US)

(21) Appl. No.: 19/038,126

(22) Filed: Jan. 27, 2025

Related U.S. Application Data
(60) Provisional application No. 63/625,579, filed on Jan. 26, 2024.

(51) Int. Cl. G01N 21/78; C12Q 1/34; C12Q 1/6844; C12Q 1/689; G01N 21/77

(52) U.S. Cl. CPC G01N 21/78; C12Q 1/34; C12Q 1/6844; C12Q 1/689; G01N 2021/7759; G01N 2333/922; G01N 2333/938

(57) ABSTRACT

Swift and accurate on-site identification of genomes of pathogens is crucial to curb its spread and contamination. In embodiments disclosed herein, a synthetic biology cascade reaction is presented on a paper substrate using CRISPR-Cas12a and recombinase polymerase amplification (RPA), enabling the programming of a standard toehold RNA switch for a genome of choice through insertion of meticulously designed an oligonucleotide (probe AB).

### CROSS-REFERENCE TO RELATED APPLICATIONS

This application claims the benefit of the filing date of U.S. Provisional Application No. 63/625,579 filed Jan. 26, 2024. The content of this earlier filed application is hereby incorporated by reference herein in its entirety.

### BACKGROUND OF THE INVENTION

The prevalence of boundless and ever-evolving pathogens, as demonstrated by increased incidences of antimicrobial-resistant infections, poses a significant threat to human health and the economy. Despite an improved understanding of the pathogenicity, the most favorable approach to manage these disease-causing agents remains to be their early detection. A timely intervention can prevent the spread of the disease and significantly minimize public harm and economic costs. This warrants the continued push for developing diagnostic techniques that can rapidly, effectively, and selectively detect pathogens at a minimal cost.

Among food-borne pathogens, Salmonella is the most dominant and widespread cause of contamination and infection. In a 2016 report, the U.S. Centers for Disease Control and Prevention estimated that 1.35 million cases and 420 deaths occur annually in the U.S. due to salmonellosis. Meanwhile, the World Health Organization reports that 1.3 billion cases of gastroenteritis and 3 million deaths are caused by Salmonella infections annually across the globe. Salmonella is a gram-negative bacterium that can contaminate a variety of food products like lettuce, milk, and poultry and has diverse means of transmission. Thus, to combat this public health and economic crisis, there is a need for diagnostics that have a short detection time frame and can be used for on-site detection to prevent the aggravated spread of Salmonella.

### BRIEF SUMMARY OF THE INVENTION

Embodiments disclosed herein are directed to kits, devices, and methods for visually detecting an organism's genome, a pathogen's genome, a Salmonella genome, and the like, on a paper substrate. According to one embodiment the method comprises: a) applying a sample that might contain the genome to the paper substrate; b) performing a cascade reaction including a CRISPR-Cas12a reaction utilizing a CRISPR-Cas12a-crRNA complex on the paper substrate; c) conducting a toehold RNA cascade reaction on the paper substrate; and d) visually detecting the presence of Salmonella genome based on the results of steps a), b) and c). According to embodiments the CRISPR-Cas12a reaction comprises: a) amplifying target sequences from the genome or a Salmonella genome when present in the sample using recombinase polymerase amplification (RPA) with a plurality of specific primers to create a plurality of amplicons; and b) activating the Cas12a-crRNA complex with the amplicons, wherein the crRNA is designed to complementary bind to the amplicons and activate Cas12a-crRNA complex.

According to other embodiments the toehold RNA cascade reaction of the method comprises: a) degrading a DNA probe (probe AB) by the activated Cas12a-crRNA complex that is designed to complementary bind to a RNA probe (probe T); b) binding of the probe T designed to complementary bind to a toehold switch in the absence of the degraded probe AB; and c) activating the toehold switch, resulting in translation of a downstream LacZ gene and β-galactosidase synthesis. In ebodiments disclosed herein the β-galactosidase synthesis provides hydrolyzing a chromogenic substrate Chlorophenol Red-β-D-galactopyranoside (CPRG) resulting in a visible color change. According to embodiments the Salmonella genome can be detected from about 100 copies or more of the genome from a Salmonella pathogen, and the crRNA can be specific for detecting S. Enteritidis (crRNAEnte) or S. Typhi (crRNATyphi). Further embodiments are directed to the method's ability to separately detect S. Typhimurium and S. Enteritidis targets.

Further embodiments are directed to a paper-based diagnostic device for visually detecting a genome, pathogenic genome, or a Salmonella genome. According to embodiments the device comprises: a) a paper substrate containing freeze-dried components for a cascade reaction including CRISPR-Cas12a reaction and toehold RNA cascade reaction; b) a Cas12a-crRNA complex wherein the crRNA is designed complementary to bind to the genome, pathogenic genome, or a Salmonella genome amplicons; c) a toehold switch linked to a LacZ gene; and d) a chromogenic substrate for β-galactosidase. Embodiments disclose that the freeze-dried components include at least one recombinase polymerase amplification (RPA) reagents, a Cas12a protein, a crRNA, a DNA probe AB, an RNA probe T, and at least one component for cell-free protein synthesis. Embodiments disclose that the device's chromogenic substrate is Chlorophenol Red-β-D-galactopyranoside (CPRG).

Additional embodiments disclosed herein are directed to a kit for visually detecting a genome, a pathogen genome, or Salmonella genome. The kit comprises: a) the paper-based diagnostic device, b) instructions for use, and c) positive and negative control samples. According to embodiments the kit further comprising specific primers for amplifying Salmonella genome targets, and the kit is capable of detecting multiple Salmonella serotype, including S. Enteritidis and S. Typhi.

Other embodiments are directed to methods for a rapid field-deployable detection of Salmonella pathogens. The method comprises: a) applying a sample suspected of containing Salmonella to the paper-based diagnostic utilizing the aforementioned device, b) incubating the device at a temperature suitable for RPA, CRISPR-Cas12a reaction, and toehold RNA cascade reaction; and c) visually observing a color change indicating the presence of Salmonella genome. The method utilizes an incubation temperature is between 37° C. and 42° C., and the incubation time is less than 2 hours.

Further embodiments disclosed herein are directed to a method for visually detecting a genome. The method comprising: a) applying a sample that might contain the genome to a paper substrate; b) performing a cascade reaction including a CRISPR-Cas12a reaction on the paper substrate; c) conducting a toehold RNA cascade reaction on the paper substrate; and d) visually detecting the presence of a genome based on the results of steps a), b), and c). According to embodiments the CRISPR-Cas12a reaction comprises: a) amplifying a target sequence of the genome when present in the sample using recombinase polymerase amplification (RPA) with specific primers to create amplicons; b) activating a Cas12a-crRNA complex with the amplicons noting that the crRNA is specific to the amplicons; c) degrading a DNA probe AB complementary to a RNA probe T by the activated Cas12a-crRNA complex; d) binding RNA probe T in the absence of DNA probe AB to a toehold switch and activating translation of a LacZ gene; e) synthesizing β-galactosidase; and f) hydrolyzing a chromogenic substrate by β-galactosidase. The chromogenic substrate is Chlorophenol Red-β-D-galactopyranoside (CPRG) and the visual detection is performed by observing a color change on the paper substrate. According to an embodiment of the method the genome is a pathogen genome in the sample.

### SEQUENCE LISTING

The specification further incorporates by reference the Sequence Listing submitted herewith via Patent Center. The Sequence Listing.xml file, identified as 010-24-13US01_SEQ, is 3,889 bytes in size and was created on Aug. 21, 2025. The Sequence Listing, electronically filed herewith, does not extend beyond the scope of the specification, and does not contain new matter.

### BRIEF DESCRIPTION OF THE DRAWINGS

FIG. 1 presents a schematic illustration of yellow-to-red color change with probe T using toehold switch and paper as the platform. FIG. 1A presents an illustration of a probe T binding to the toehold switch to initiate repressed translation, and induces the color change. FIG. 1B presents the color transition on the paper discs, as recorded using a phone camera, and FIG. 1C presents a graph quantifying the color change using absorbance spectroscopy at λ_{570 nm}. Experiments were performed in triplicate. Data are represented as mean±S.D. The two-tailed P-value equals 0.0005 (n=3). By conventional criteria, this difference is considered to be statistically significant.

FIG. 2A and FIG. 2C present a free probe T activation of the toehold switch and induces color change, respectively. FIG. 2B and FIG. 2C present inhibited probe T (probe AB-T hybrid) does not activate. FIG. 2C presents probe AB alone (complementary to probe T) and blank (no DNA or RNA) that are used as negative controls and show no color change. The color change on the paper discs was photographed using a microscope at 2× magnification and quantified using absorbance spectroscopy at λ₅₇₀ₙₘ. Experiments were performed in triplicate. Data are represented as mean±S.D. The two-tailed P-value equals 0.0005 (n=3). By conventional criteria, this difference is considered to be statistically significant.

FIG. 3A presents a graph of target induced color change upon CRISPR and toehold cascade reaction, while its absence does not. FIG. 3B presents a graph of crRNA in the CRISPR Cas12a complex of the diagnostic assay that is programmed for S. Typhimurium, the color change is observed only with the intended target S. Typhimurium biomarkers (ssDNA_{Typhi} and dsDNA_{Typhi}), but not with non-target S. Enteritidis. FIG. 3C presents a graph that when crRNA is programmed for S. Enteritidis, color change is observed only for ssDNA_{Ente} and dsDNA_{Ente}. Experiments were performed in triplicate. Data are represented as mean±S.D. The two-tailed P-value is less than 0.0001 (n=3). By conventional criteria, this difference is considered to be statistically significant.

FIG. 4 presents CRISPR and toehold reaction using 0.05, 0.5, 5, 50, 500 nM of ds DNA_{Typhi} (amplicons). Paper discs were photographed at 2×magnification, and color change was quantified at, λ₅₇₀ₙₘ. Experiments were performed in triplicate. Data are represented as mean±S.D. The two-tailed P-value equals 0.0024 (n=3). By conventional criteria, this difference is considered to be statistically significant.

FIG. 5A presents detection of various copy numbers of S. Typhimurium genome. The two-tailed P-value equals 0.0053. By conventional criteria, this difference is considered to be statistically significant. FIG. 5B presents 400 copies of S. Typhimurium genome in milk and lettuce matrices. The two-tailed P-value equals 0.0002 and less than 0.0001, respectively. By conventional criteria, this difference is considered to be statistically significant. Experiments were performed in triplicate. Data are represented as mean±S.D. Paper discs were photographed at 2× magnification, and color change was quantified at λ₅₇₀ₙₘ.

FIG. 6 presents a schematic illustration of toehold switch activation for translation with trigger RNA. Trigger RNA (probe T) binds to toehold RNA and induces a conformation change, and thereby allows ribosome binding and initiation of translation.

FIG. 7 presents a schematic illustration of the paper-based sensor for target DNA detection. As presented in FIG. 7A the target binds to crRNA and activates Cas12a to cleave probe AB, making probe AB unavailable for probe T hybridization. The free probe T binds to toehold switch and initiates protein translation and color change of CPRG substrate on paper. As presented in FIG. 7B the absence of target DNA, Cas12a is unable to cleave probe AB, which then hybridizes to and inhibits probe T from binding and activating toehold switch. Thus, toehold switch remains inactive, unable to initiate translation and color change.

FIG. 8 presents a schematic illustration of protocol for detecting whole Salmonella genome on paper. RPA generates amplicons from a highly conserved sequence within the genome. The amplicons get recognized by crRNA, which activates Cas12a, leading to the cleavage of probe AB and making it unavailable for probe T hybridization. Free probe T then binds toehold switch, initiates repressed translation, and induces a color change.

FIG. 9 presents primer screening for S. Typhimurium genome using (FIG. 9A) different reverse primers and (FIG. 9B) different forward primers.

FIG. 10 presents the detection of various copy numbers of S. Typhimurium genome and the color change on the paper discs as recorded using a phone camera.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The “cascade reaction” disclosed herein refers to a synthetic biology process combining CRISPR-Cas12a technology with toehold RNA switches. This reaction is designed to amplify and visually signal the presence of, e.g., a genome, a pathogen genome, or Salmonella genome on a paper substrate. The cascade begins with recombinase polymerase amplification (RPA) if the genome is present. The amplified DNA is then recognized by the CRISPR-Cas12a system, which is programmed with a guide RNA to bind and cleave DNA. Upon activation, Cas12a exhibits trans-cleavage activity, indiscriminately cutting nearby DNA, as more fully described herein below. This cleavage event triggers the release of a signal (e.g., Probe T) that interacts with a toehold RNA switch, leading to a series of synthesis and hydrolysis reactions resulting in a visible color change on the paper substrate.

The CRISPR-Cas12a reaction disclosed herein is directed to a Cas12a CRISPR complex (aka Cas12a CRISPR system) that can recognize and cleave DNA sequences across a wider range of target sites with minimal adjustments to the guide RNA. The Cas12a complex is a CRISPR RNA (crRNA) guided nuclease and is a type of CRISPR-based genome editing technology where the Cas12a protein acts as an enzyme that cuts DNA at a specific location guided by the complementary sequence on the crRNA, allowing for targeted manipulation of genetic material; essentially, the crRNA directs the Cas12a enzyme to the desired spot on the DNA to cleave it. The Cas12a as disclosed herein is directed to an enzyme for DNA cleavage and detection, which has the ability to target specific DNA sequences and trigger a cascade reaction.

The term “Toehold RNA Switch” as disclosed herein is an RNA switch, which is an RNA-based regulatory element that can be programmed to respond to specific RNA or DNA sequences. The toehold RNA switch is used in the cascade reaction to differentiate between different Salmonella serotypes.

The toehold RNA cascade reaction as disclosed herein activates a toehold switch and leads to β-galactosidase synthesis and visible color change via a toehold switch, i.e., an RNA hairpin structure that contains a ribosome binding site (RBS) and start codon (AUG) in a loop and bulge, respectively. A specific trigger RNA (Probe T) binds to the toehold domain, it initiates a strand displacement reaction causing the hairpin to unfold, exposing the RBS and start codon; and this conformational change allows ribosomes to bind and initiate translation of the downstream LacZ gene. The LacZ gene encodes the enzyme β-galactosidase and the synthesized β-galactosidase can hydrolyze the chromogenic substrate Chlorophenol Red-β-D-galactopyranoside (CPRG). This hydrolysis reaction results in a visible color change on the substrate. Thus, the entire cascade, from trigger RNA binding to color change, provides a visual indication of the presence of the specific trigger RNA sequence.

The term “Recombinase Polymerase Amplification (RPA)” as disclosed herein is directed to an isothermal amplification method used to amplify DNA sequences at a constant temperature. In embodiments disclosed herein, the RPA is integrated with CRISPR-Cas12a.

The term “specific primers” as used herein are primers that are essential for obtaining amplicons from the whole genome. These primers are typically 30-35 nucleotides long and are designed to target specific sequences within the genome. The primers bind to complementary sequences in the target DNA, ensuring that only the desired regions are amplified.

The term “Paper Substrate” as disclosed herein is directed to the medium for the diagnostic assay. The paper substrate provides portability, low cost, and ease of use.

The term “Visual Detection” as disclosed herein is directed to a method of visual detection used in the disclosed assay, which produces a visible signal (a color change, e.g., from yellow to red) that can be observed without specialized equipment.

The term “Genome Detection” as disclosed herein is directed to a process for the detection of, for example, the Salmonella genomes using the CRISPR-Cas12a and toehold RNA cascade reaction.

The term RPA as disclosed herein describes Recombinase Polymerase Amplification, which is a molecular biology technique used to rapidly amplify specific DNA or RNA sequences at a constant temperature (isothermal amplification),

The term “palindromic sequence” as disclosed herein is a sequence of nucleotides in DNA or RNA that reads the same in both directions on complementary strands. Thus, in DNA, a palindromic sequence is a sequence of nucleotides that is followed by its complement sequence in reverse order. For example, the sequence GGATCC on one strand of DNA is a palindrome because the sequence on its complementary strand is CCTAGG.

β-Galactosidase, also known as lactase, as disclosed herein relates to its use to hydrolyze Chlorophenol Red-β-D-galactopyranoside (CPRG), causing a visible color change from yellow to dark red, is a commonly used application for detecting β-galactosidase activity in research settings.

The term complementary as used herein means that the amplicons have a nucleotide sequence that perfectly matches, base-pair by base-pair, with the sequence of a specific RNA molecule, allowing them to bind together.

The term serotype (or serovar) as used herein is a distinct variation within a species of bacteria (e.g., Salmonella) or virus or among immune cells of different individuals.

The term sensor used herein refers to the system starting from CRISRP-cas12a complex recognizing the amplicons of a target genome is the universal sensor.

### DETAILED DESCRIPTION OF THE INVENTION

Salmonella, the most prevalent food-borne pathogen, poses significant medical and economic threats. Swift and accurate on-site identification and serotyping of Salmonella is crucial to curb its spread and contamination. We utilize a synthetic biology cascade reaction on a paper substrate using CRISPR-Cas12a and recombinase polymerase amplification (RPA), enabling the programming of a standard toehold RNA switch for a genome of choice. This approach employs just one toehold RNA switch design to differentiate between two different Salmonella serotypes, i.e., S. Typhimurium and S. Enteritidis, without the need for reengineering the toehold RNA switch. The sensor exhibits high sensitivity, capable of visually detecting as few as 100 copies of the whole genome from a model Salmonella pathogen on a paper substrate. Furthermore, this robust assay is successfully applied to detect whole genomes in contaminated milk and lettuce samples, demonstrating its potential in real sample analysis. Due to its versatility and practical features, genomes from different organisms can be detected by merely changing a single RNA element in this universal cell-free cascade reaction.

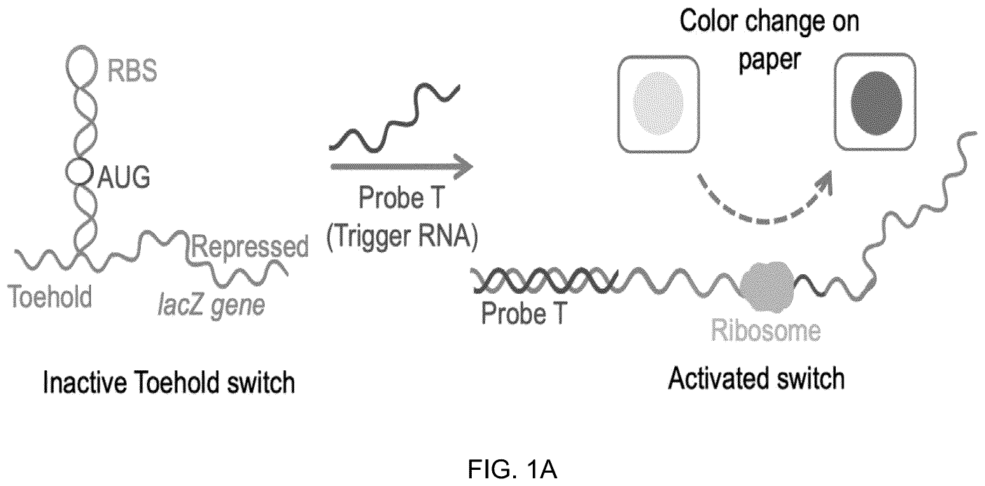

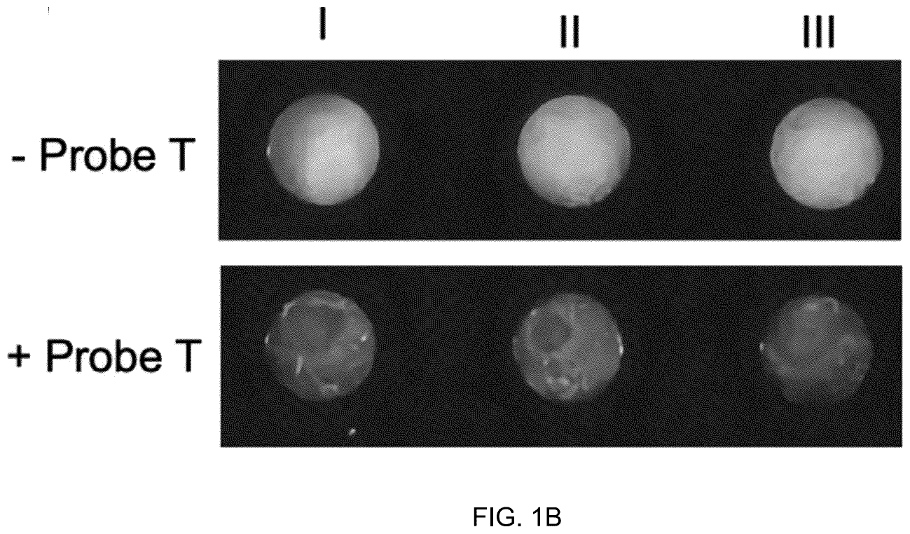

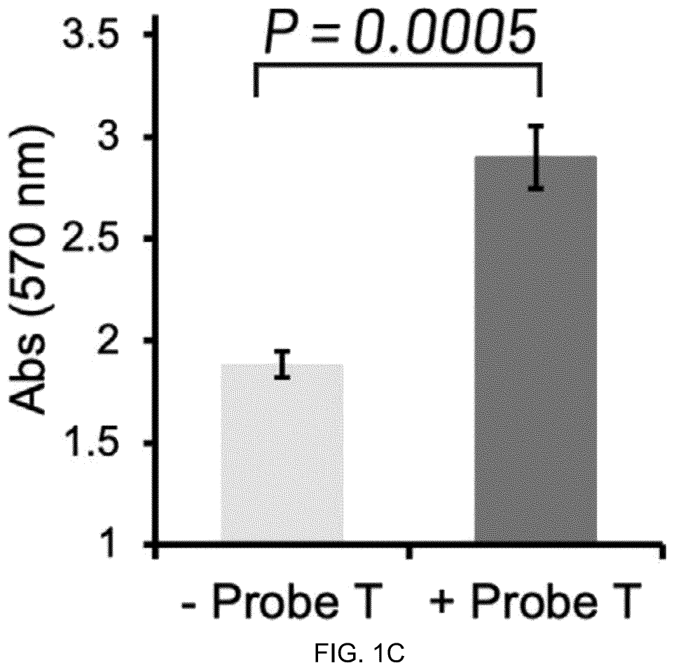

Progress in synthetic biology, i.e., the engineering of novel gene circuits and dynamic sensors, has driven efforts for innovative diagnostic tools and molecular sensing mechanisms. Most notably, Green et al. have demonstrated the successful integration of strand displacement interactions with synthetic riboregulatory in the “toehold switches” that activate protein translation in response to a specific RNA sequence. In the absence of this “trigger RNA,” the ribosome binding site (RBS) and start codon (AUG) are sequestered due to the secondary structure of the switch (FIG. 1A). Hybridization to the trigger RNA, however, initiates a conformation change that frees up the RBS and AUG and thereby promotes translation (FIG. 1B), e.g., translation of lacZ gene and synthesis of P-galactosidase. Pardee et al. have used toehold RNA switches for the development of Zika and Ebola virus sensors. Takahashi et al., on the other hand, have utilized toehold switches for gut microbiome characterization and RNA quantification in stool samples.

Embodiments disclosed herein are directed to a highly modular paper-based diagnostic assay for the visual detection of a model bacterial genome using a synthetic biology cascade reaction.

While these toehold switch designs have greater dynamic range than conventional RNA switches, their construction is difficult. Design packages can help predict the RNA structure of toehold switches, yet very few show high experimental performance. Hence, the use of these switches is laden with iterative design and refinement processes prior to its effective deployment. Furthermore, once successfully engineered, the switch can exclusively detect a specific RNA strand. A different RNA would require structuring a completely new toehold switch. Hence, it is highly desirable to develop a universal design that can not only detect a specific RNA strand but can also be broadly applied to detecting any nucleic acid of interest, short or long, single-stranded or double-stranded. This further facilitates whole genome detection efforts without redesigning a new toehold switch for each target. To expand its utility beyond the detection of a specific RNA, we have developed a cascade reaction here by combining a previously tested and optimized toehold switch design with CRISPR-Cas12a.

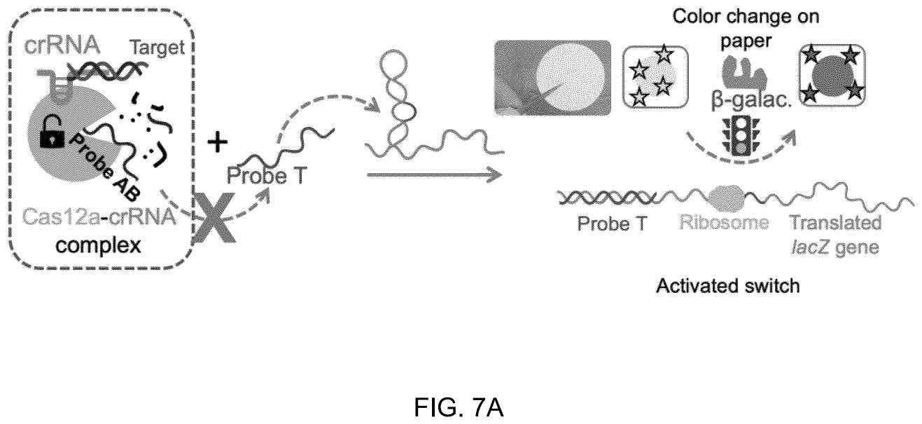

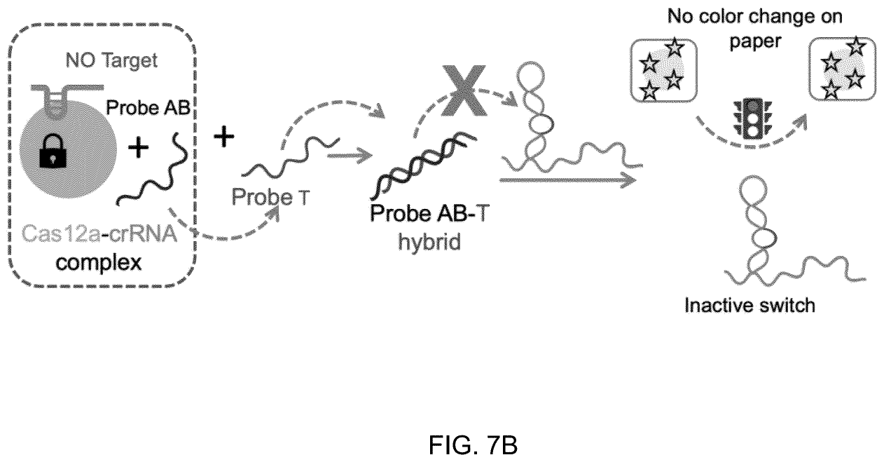

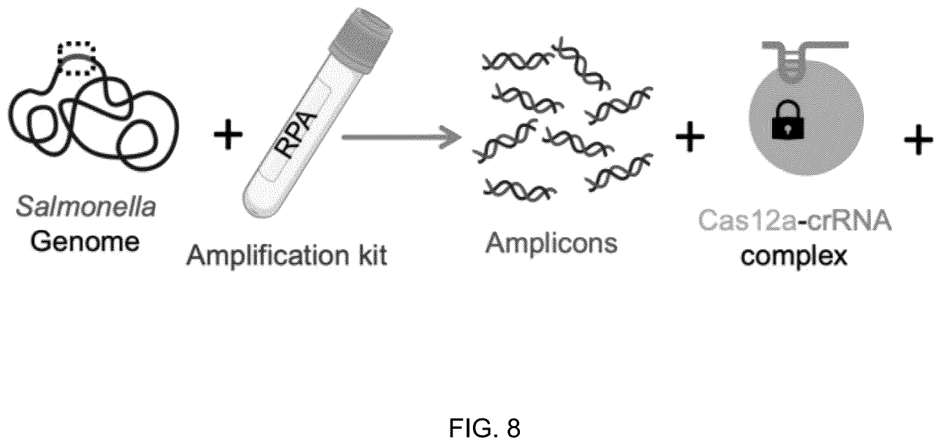

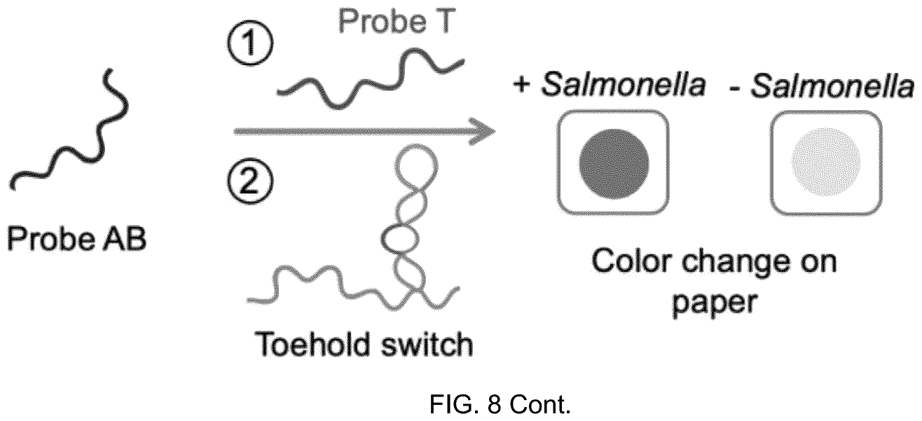

Clustered regularly interspaced short palindromic repeats (CRISPR) and the associated Cas12a complex comprise of a CRISPR RNA (crRNA) guided nuclease that provides indiscriminate DNA cleavage activity to any DNA strands in its vicinity when activated. This cleavage activity is initiated by a specific target DNA that is complementary to the crRNA. The cascade reaction utilizes recombinase polymerase amplification (RPA) to create amplicons from the whole genome using specific primers. The crRNA in the Cas12a-crRNA complex is designed to be specific to the amplicons. The target amplicons bind and activate in the Cas12a-crRNA complex which degrades the probe AB (probe AB being complementary to probe T) in the assay. If not degraded probe AB inhibits the toehold's function by binding probe T, i.e., probe AB-probe T is a DNA: RNA hybrid. Since there is no probe AB left to, e.g., bind to inhibit probe T, the probe T binds/complexes to the toehold switch and activates it. This activation will result in the successful translation of the downstream LacZ gene and β-galactosidase synthesis. Consequently, β-galactosidase mediates the hydrolysis of a chromogenic substrate, Chlorophenol Red-β-D-galactopyranoside (CPRG, yellow-colored) (e.g., see FIG. 7A), resulting in a yellow to dark red color transition.)

Cas12a-based diagnostics are highly programmable through the replacement of crRNA in the Cas12a crRNA complex. Thus, we first integrated CRISPR-Cas12a with the toehold switch for the detection of a DNA target of choice, e.g., a Salmonella serotype. Briefly, when the target DNA (target) is present in the environment, it activates the Cas12a-crRNA complex and results in trans-cleavage of probe AB (i.e., DNA, FIG. 7A), which will be unable to hybridize with and inhibit probe T (trigger RNA). The free probe T can then bind to and activate the toehold switch. This activation will result in the successful translation of the downstream lacZ gene and β-galactosidase synthesis. Consequently, β-galactosidase mediates the hydrolysis of a chromogenic substrate, Chlorophenol Red-β-D-galactopyranoside (CPRG, yellow colored) (FIG. 7A), resulting in a yellow to dark red color transition.

On the other hand, when the target DNA is absent, Cas12a remains inactive and cannot cleave probe AB, which can hybridize with probe T. Since probe T is no longer free, but in a DNA-RNA hybrid form (probe AB-T hybrid), it cannot activate toehold switch and promote the translation and production of LacZ. Hence, no color change can be observed (FIG. 7B). Running the reaction on a paper disc makes it possible to observe the reaction progress and result on a solid substrate. Here, this synthetic biology cascade reaction is evaluated for paper-based detection of a genome from a specific food-borne pathogen, Salmonella.

First, we tested the compatibility of the toehold reaction on a paper substrate (FIG. 1). To a bovine serum albumin (BSA)-treated filter paper, we added the reaction components required for protein synthesis or expression of LacZ enzyme, CPRG, and the isolated toehold RNA (FIG. 1A). Upon the addition of probe T, the color of CPRG on the paper substrate turned from yellow to dark red (performed in triplicate, FIG. 1B, and FIG. 1C). The toehold RNA has the genetic information to synthesize the protein that changes the color. Thus, probe T controls toehold RNA function by binding to it. Without probe T, no color change is observed, as expected.

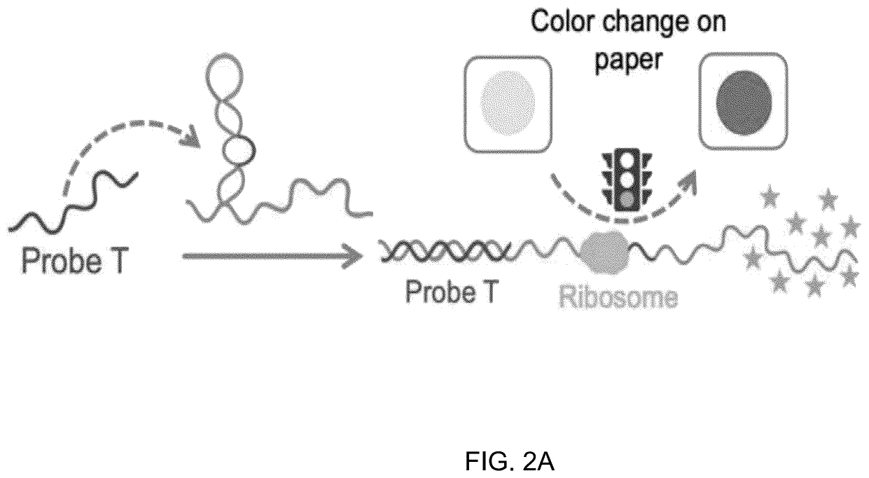

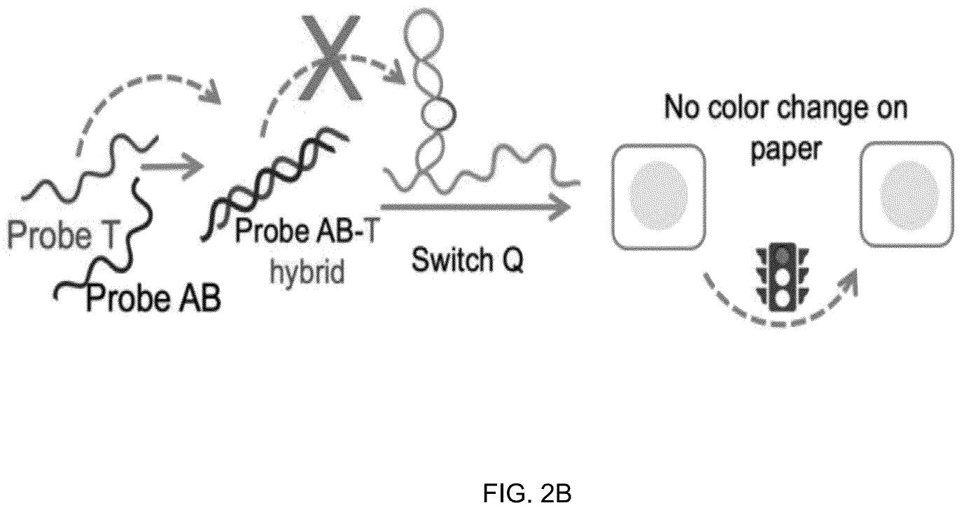

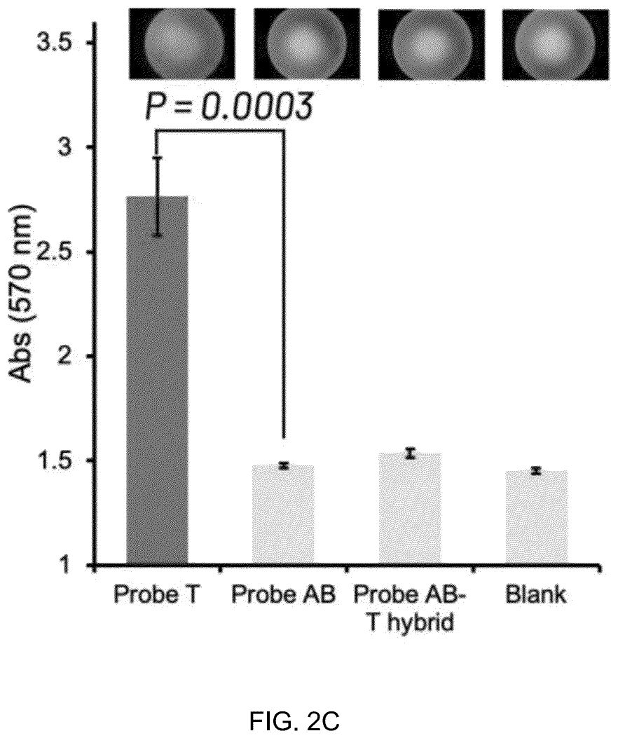

Further, we checked whether free and inhibited probe T could initiate the toehold reaction (FIG. 2). We ran the reaction with free probe T (FIG. 2A) and probe AB-T hybrid (FIG. 2B). Probe AB by itself and blank (buffer only) were used as negative controls. As anticipated, only free probe T could start the translation process and result in the yellow-to-red color change (FIG. 2A; and FIG. 7A). Alternatively, the probe AB-T hybrid (inhibited probe T) was unable to initiate the protein synthesis process (FIG. 7B). These results are extremely consequential, allowing us to proceed to detect genomic targets of choice through the integration of CRISPR-Cas12a prior to the toehold reaction (FIG. 7A).

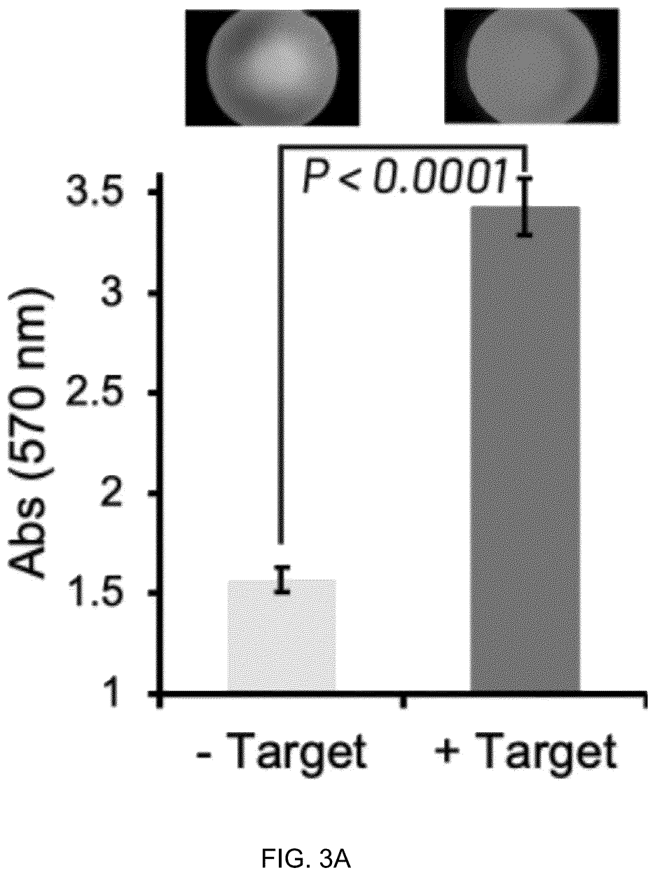

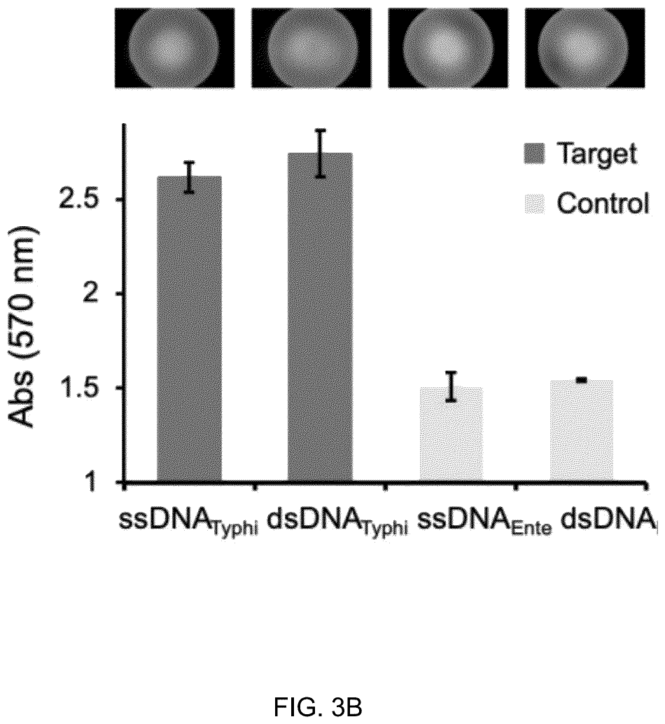

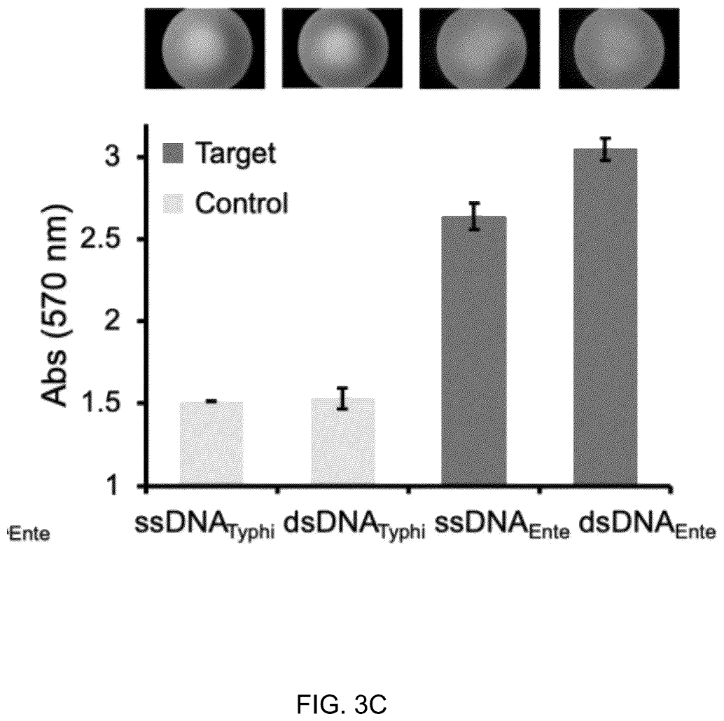

To test our assay with Cas12a, we identified a short dsDNA fragment from the conserved genomic region of a Salmonella serotype, S. Typhimurium (tDNA_{Typhi} in Table 1, below). Parallelly, we designed a crRNA (crRNA_{Typhi}) against this target (Table 1) Briefly, the target DNA was added into the reaction assay containing Cas12a-crRNA and probe AB mixture. After the degradation of probe AB by activated Cas12a (1 h), probe T was added to the reaction. Since there is no probe AB left to inhibit probe T, the toehold reaction is activated, resulting in a yellow-to-red color transition (FIG. 3a). This result was not observed when the target DNA was absent.

Table 1 provides a list of oligonucleotide sequences used. For crRNA, the recognition region is in bold and underlined. For truncated Salmonella targets, target recognition site is in bold and underlined, with PAM highlighted in yellow.

| Label | Sequence |
|---|---|
| Probe T (Trigger RNA) | 5′-GAC ACA GGA CAU GAA ACU GAU GAG AAU AGA GCG |
| | AAA-3′ |
| Probe AB (DNA | 5′-TTT CGC TCT ATT CTC ATC AGT TTC ATG TCC TGT GTC-3′ |
| complementary to probe | |
| T) | |
| 1F (forward primer 1) | 5′-CTC TTC GGC ACAAGT AAT ATC AAC GGT ACG-3′ |
| 2F (forward primer 2) | 5′-CTC CGC AAG TTG AGC TTT TTC CAG ATC TTC AC-3′ |
| 3F (forward primer 3) | 5′-CAC AAG TAA TAT CAA CGG TAC GGT CTC TGT AG-3′ |
| 4F (forward primer 4) | 5′-CTC CGC AAG TTG AGC TTT TTC CAG ATC TTC-3′ |
| 1R (reverse primer 1) | 5′-TCA GTA TTG AGG AAAAAG AAG GGT CGT CGT TAG-3′ |
| 2R (reverse primer 2) | 5′-CAG TAT TGA GGA AAA AGA AGG GTC GTC GTT AGG-3′ |
| 3R (reverse primer 3) | 5′-GAG CAG CCG CTC AGT ATT GAG GAAAAA GAA G-3′ |
| 4R (reverse primer 4) | 5′-CAG TAT TGA GGA AAA AGA AGG GTC GTC GTT AG-3′ |
| crRNA_{Typhi} | 5′-UAA UUU CUA CUA AGU GUA GAU UCG AGA UCG CCA |
| | AUC AGU CC-3′ |
| crRNA_{Ente} | 5′-UAA UUU CUA CUA AGU GUA GAU CGA AUA ACA UCC |
| | UCA ACU UC-3′ |
| tDNA_{Typhi} | 5′-TCG TCG TTA GGA CTG ATT GGC GAT CTC GAT AAA |
| (truncated S. | GTC TCT ACA GAG ACC GT-3′ |
| Typhimurium target i.e., | |
| ssDNA_{Typhi}) | |
| CDNA_{Typhi} | 5′-ACG GTC TCT GTA GAG ACT TTA TCG AGA TCG CCA |
| (complementary to | ATC AGT CCT AAC GAC GA-3′ |
| tDNA_{Typhi}) | |
| tDNA_{Ente} | 5′-GGT ATC TGC TGA AGT TGA GGA TGT TAT TCG CAA |
| (truncated S. Enteritidis | AGG GAT CCG TCA GAC CT-3′ |
| target i.e., ssDNA_{Ente}) | |
| CDNA_{Ente} (complementary | 5′-AGG TCT GAC GGA TCC CTT TGC GAA TAA CAT CCT |
| to tDNA_{Ente}) | CAA CTT CAG CAG ATA CC 3′ |
| ssprobe | 5′-/56-FAM/ TTTTTT TTT/31ABkFQ/-3′ |

Along with S. Typhimurium, S. Enteritidis is a prominent contributor to salmonellosis in humans. The distinction between the strain types can prove helpful in tracking the origin of an outbreak and the clinical treatment of an infection. With that in mind and to highlight the modular and programmable advantage of our protocol, we designed a crRNA for S. Enteritidis (crRNAEnte). First, using crRNA_{Typhi}, we performed a Cas12a reaction using S. Typhimurium and S. Enteritidis targets separately. The reactions are done against single (ss) and double (ds) stranded targets. Here, since the crRNA was designed specifically for S. Typhimurium, the corresponding color change occurred for only S. Typhimurium targets (FIG. 3B). On the other hand, by simply swapping out crRNA_{Typhi} with crRNA_{Ente}, we were able to explicitly detect the S. Enteritidis targets (FIG. 3C). The results indicate that by merely changing the crRNA content, the diagnostic assay can be quickly reprogrammed for another serotype. With the inclusion of a meticulously thought out CRISPR step in the toehold reaction, we can eliminate the arduous and intricate process of designing a new toehold switch for every potential new target of interest. As such, just one toehold switch design can be employed for any target of interest.

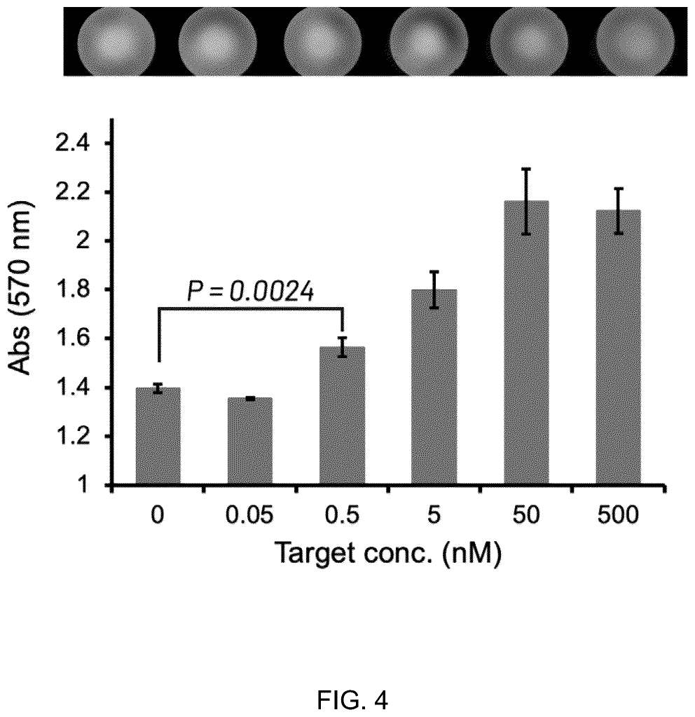

To evaluate the detection capacity, we ran a sensitivity test for our approach. We tested different concentrations of S. Typhimurium target (dsDNA_{Typhi}, 0.05, 0.5, 5, 50, and 500 nM) and performed the entire reaction protocol. While there was an increase in color variance with increasing target concentration, we could confidently quantify up to 0.5 nM of DNA (FIG. 4).

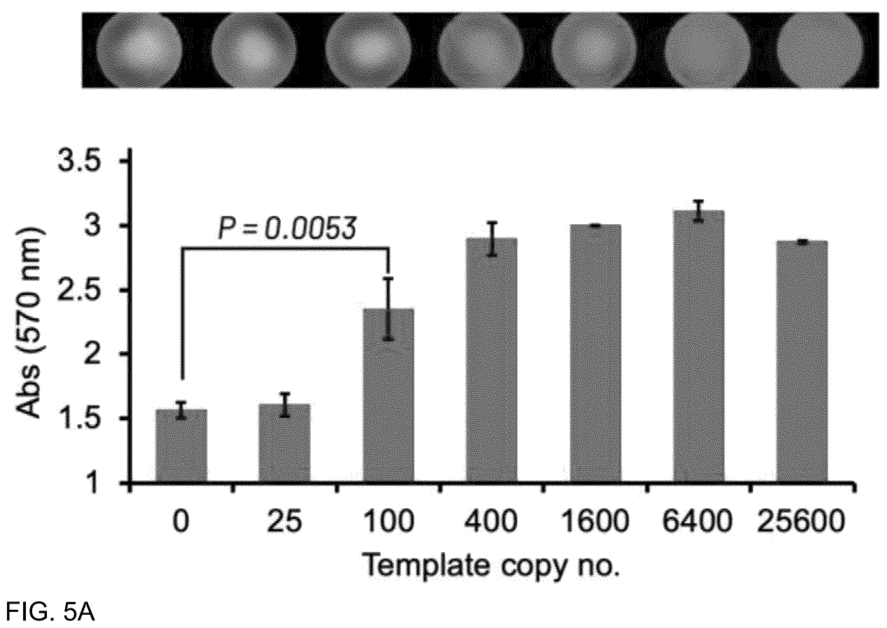

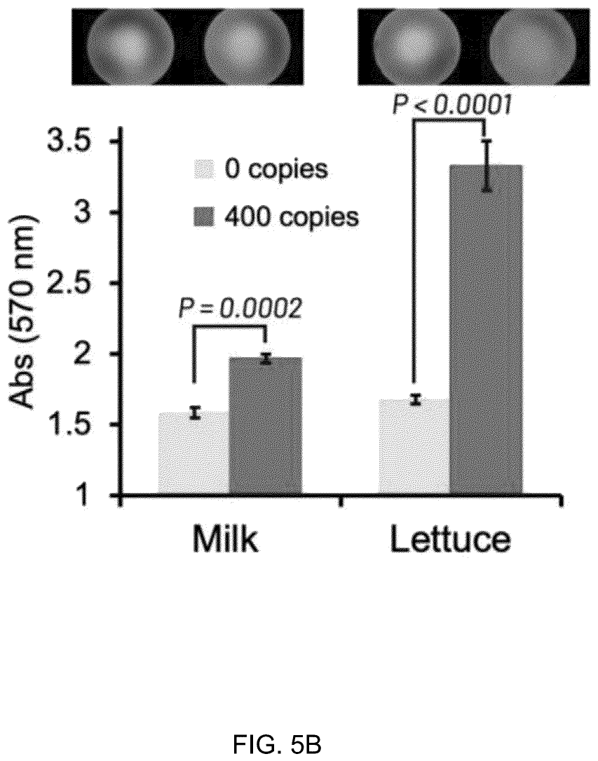

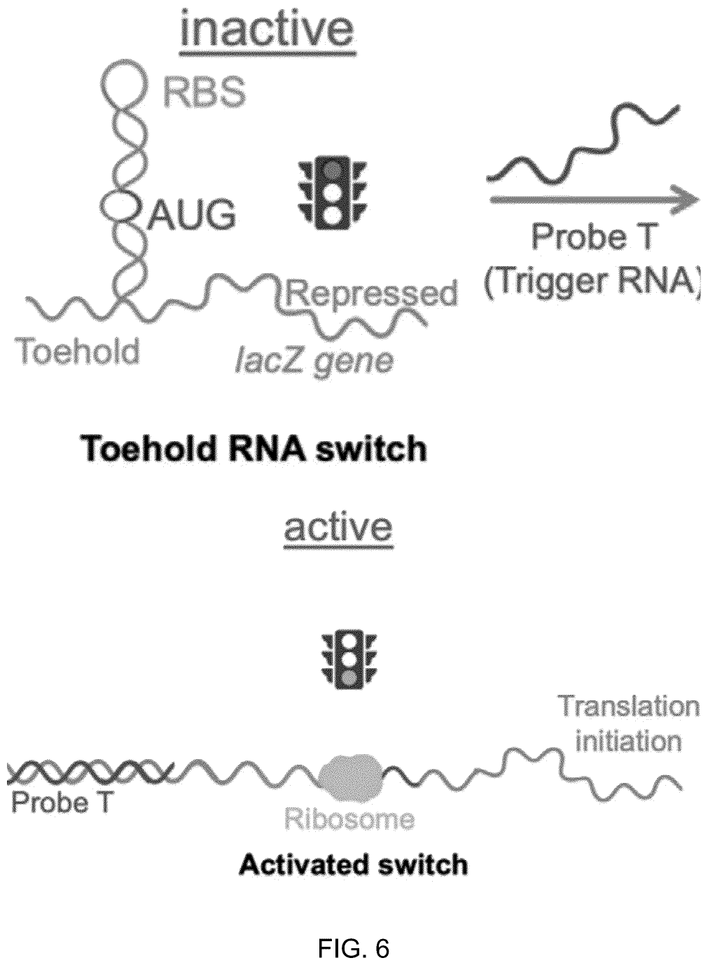

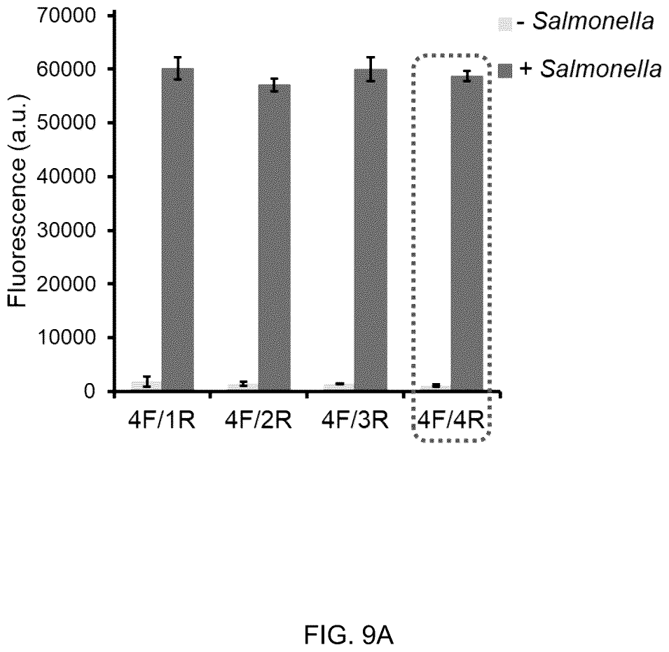

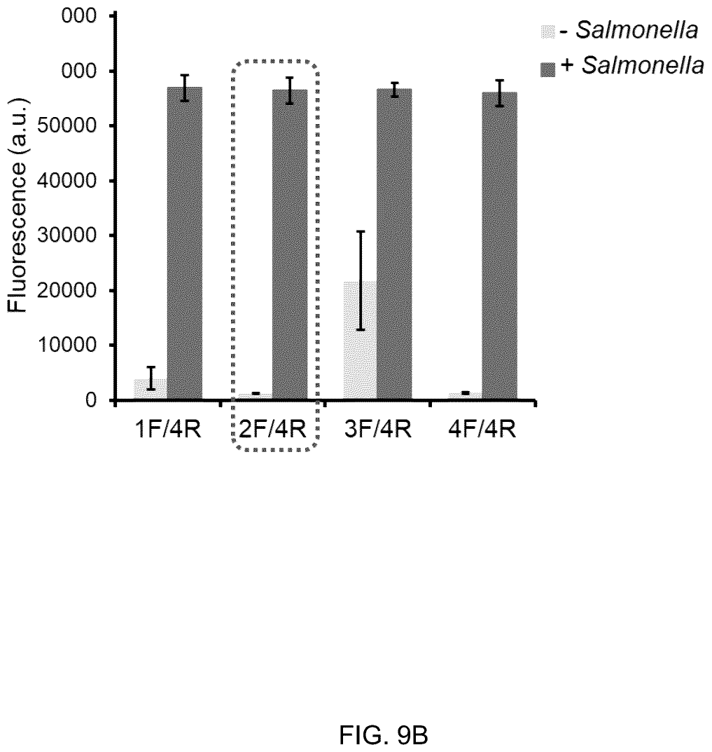

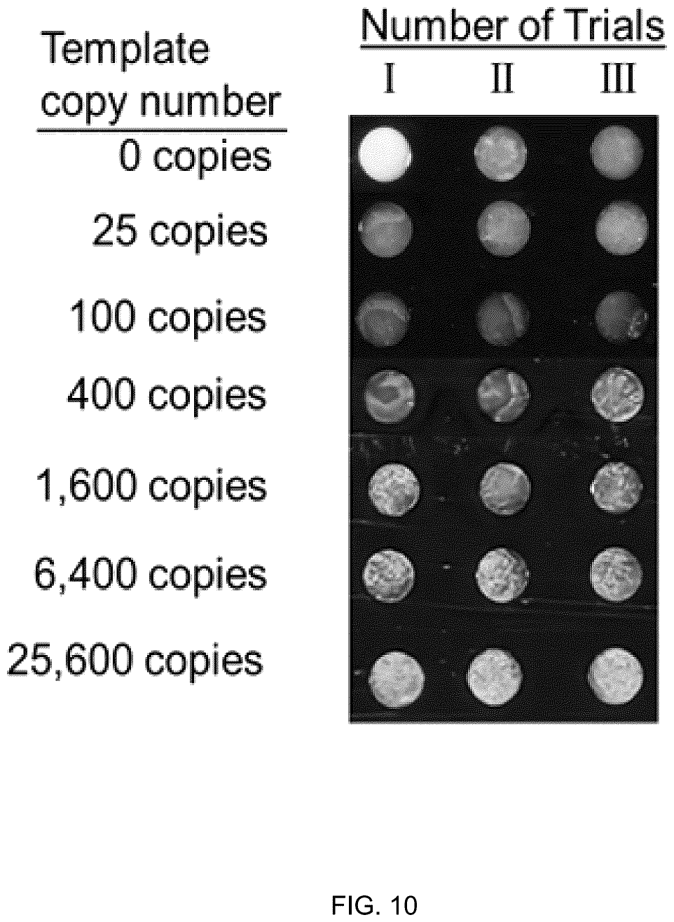

We next tested our technology for the detection of whole genome of S. Typhimurium and improved the sensitivity through inclusion of an isothermal amplification reaction. Recombinase polymerase amplification (RPA) was used to generate the conserved sequence within the S. Typhimurium genome after screening for various reverse and forward primers (FIG. 9). The studies were performed with a wide range of genomic copies (0, 25, 100, 400, 1600, 6400, and 25600), and unexpectedly, even 100 copies of the genome could be identified with a bright yellow-to-red color change within 4 h (FIG. 5A; and FIG. 10). The lower detection limit in this study is attributed to insufficient target for initiating the cascade reaction whereas the upper limit is due to color saturation at higher target amounts.

### EXAMPLES

The present invention is illustrated by the following examples. However, it should be understood that the invention is not limited to the specific details of these examples.

Materials and Methods: The toehold switch (ZIKV_Sensor_2′B_LacZ; plasmid #′75006) was purchased from Addgene (Watertown, MA, USA). The template S. Typhimurium genome was purchased from American Type Culture Collection (ATCC; Manassas, VA, USA). All other oligonucleotides were purchased from Integrated DNA Technologies, Inc. (Coralville, IA, USA). The RPA kit was purchased from TwistDx Limited (Cam-bridge, UK). PURExpress In Vitro Protein Synthesis Kit, Monarch PCR & DNA Cleanup Kit, and EnGen Lba Cas12a (Cas12a) were purchased from New Eng-land Biolabs (Ipswich, MA, USA). BSA and magnesium chloride hexahydrate were purchased from Amresco (Solon, OH, USA). Purelink Genomic DNA extraction kit, fetal bovine serum, LB agar plate with 50 μg mL⁻¹ Kanamycin, and RNase-free dis-tilled water were purchased from Thermo Fisher Scientific (Waltham, MA, USA). Kanamycin Sulfate, LB agar plate with 50 μg mL⁻¹ Kanamycin, ZymoP-URE II Plasmid Maxiprep Kit, Integra 2 mm biopsy punch, 96-well half-area plate, and were purchased from VWR International (Secaucus, NJ, USA). Chlorophenol Red-P-D-galactopyranoside, i.e., CPRG, Whatman filter paper, Protector RNase Inhibitor, glycerol, and all other chemicals were purchased from Sigma-Aldrich (St. Louis, MO, USA). Whole milk and green leaf lettuce were sourced from the local grocery store.

Toehold Switch Isolation: Glycerol stocks were prepared for long-term storage of the toehold switch plasmid obtained from Addgene. The bacterial solution was then streaked on LB agar plates having 50 μg mL⁻¹ Kanamycin and incubated at 37° C. overnight. A starter culture of 5 mL LB Broth (50 μg mL⁻¹ Kanamycin) was inoculated with a single colony of bacteria and incubated in an incubator shaker at 37° C. and 220 rpm for a minimum of 10 h. For the main culture, 1/500 volume of starter culture was added to LB Broth (+50 μg mL⁻¹ Kanamycin), e.g., 0.5 mL of starter culture added to 250 mL of LB Broth, and incubated in a shaker at 37° C. and 200 rpm for at least 20 h. The ZymoPURE extraction protocol for low-copy number was followed for isolation of the plasmid. Monarch Cleanup kit was used to further concentrate the isolated toehold plasmid. Nanodrop Spectrophotometer was used to confirm that toehold concentration was greater than 50 nM, and then this was stored at −20° C. for all future experiments.

Designing Primers, Truncated Target, and crRNA for Salmonella: The RPA primers were designed by whole genome alignment of AE006468, CP110657.I for S. Typhimurium. The sequence alignment tool in Snap-Gene software (from Insightful Science; available at snapgene.com) was used for the genome alignment. A highly conserved 24 base sequence in the invA gene and having a PAM sequence of TTTA was selected as the target sequence to be amplified. Nucleic acid amplification strategies like RPA are prone to false positive results because of nonspecific off-target amplicons being generated from interfering DNAs. To address the possibility of this systemic error, four pairs of primers were screened to ensure the amplification of a very specific target. These four sets of forward and reverse primers were designed for the aforementioned 24 nucleotides long target site. All crucial features of the primers mentioned in the TwistDx de-sign manual were retained. The crRNA for the identification of the target amplicon was designed with a recognition sequence that was complementary to the target sequence, and a scaffold that associates with the Casl2a enzyme.

Similarly, for S. Enteritidis, AM933172, NC_011294, and CP050723 were aligned, and a 24 base consensus sequence (in the invA gene) having PAM sequence of TTTG was selected as the target region. The crRNA for this target was designed similarly. All nucleotide sequences are listed in Table 1 of the Supporting Information.

Oligonucleotide Preparation: As per the ATCC certificate of analysis, 17 μg of S. Typhimurium genome was dissolved in 250 μL of RNase-free DI water (RDI) to form a 1×solution having 1.28×10⁷ copies μL⁻¹. All other oligonucleotides were dissolved in RDI to form 1 mM stock solutions. These 1 mM stocks were diluted using RDI to form 100 μM stocks, and their concentration was confirmed using Nanodrop Spectrophotometer. The 100 μM oligonucleotides were further diluted to form 10 μM solutions. 10 μM of all primers were prepared in RDI. 10 μM of crRNA was prepared in 1×Tris-EDTA buffer, pH 7.9 at 25° C. 10 μM of tDNA_{Typhi}, CDNA_{Typhi}, tDNA_{Ente}, and cDNA_{Ente} was prepared in cas buffer—10 mM Tris-HCl, 50 mM NaCl, 10 mM MgCl2, 100 μg mL⁻¹ BSA, pH 7.9 at 25° C. (same as commercially available 1× NE Buffer 2.1).

Testing Efficiency of Toehold Reaction: The Whatman filter paper was first treated by submerging in a 5% BSA solution (prepared in 1×PBS buffer) on a shaker (30 rpm) for 24 h. After 24 h, the paper was rinsed twice in RDI for 10 min each. Once completely dry, the biopsy punch was used to cut out 2 mm discs and to place them in the 384-well assay plate. For cell-free protein expression, a protein master mix containing the following was prepared—0.72 μL of solution A, 0.54 μL of solution B, 0.5 μL of 40 U μL⁻¹ RNase Inhibitor, and 0.06 UL of 20 mg μL⁻¹ of CPRG. While keeping the assay plate on ice (to prevent the reaction from starting), 1.37 μL of this freshly prepared mix was added to the paper for each reaction. Next, 0.5 μL of 37.4 nM of isolated toehold switch was added to paper. Lastly, 0.5 μL of cas buffer and 0.5 μL of 0.4 μM probe T (diluted in cas buffer) were added to the paper separately. Upon addition of probe T, the plate with the paper disc was placed in a pre-heated plate reader (37° C.) and absorbance at 570 nm (λ₅₇₀ₙₘ) was measured over 2 h at 37° C. Post incubation, images of the paper were captured at 2× magnification using an Eclipse TS100 microscope with a camera attachment.

Toehold Reaction Using Different Probes: 4 μM of probe AB and 2 μM of probe T were prepared in cas buffer. Then the two were mixed to prepare probe AB-T hybrid containing 0.4 μM of probe T and 0.6 μM of probe AB. The two probes were mixed and left to hybridize at room temperature (R.T) for I h. Pre-treated paper was cut into discs and placed in the 384-well plate, then 1.37 μL of master mix and 0.5 μL of 37.4 nM toehold switch were added to the paper like before. 0.5 μL of the following was added next −0.4 μM of probe T, 0.6 μM of probe AB, probe AB-T hybrid, and cas buffer. Like the previous study, and in all the subsequent studies, all reaction components for the toehold reaction were added to the paper while the plate was on ice. Absorbance values were measured at 37° C., and images were captured using the aforementioned protocol.

Coupling CRISPR-Cas12a with Toehold Reaction: 1 μM of dsDNA_{Typhi} Was prepared by mixing equal concentrations of tDNA_{Typhi} and cDNA_{Typhi} in cas buffer. 2.4 μM of Cas12a and 2.88 μM of crRNA_{Typhi} were mixed in cas buffer and incubated at 37° C. for 30 min to form the Cas12a complex. For a 4 μL reaction, 1 μL of the cas complex (0.6 μM: 0.72 μM), 0.6 UL of 4 μM probe AB (0.6 μM), and 1.4 μL of cas buffer were added to both-1 μL of cas buffer and 1 μL of 4 nM dsDNA_{Typhi} (1 nM) in 6 PCR tubes (3 each). All tubes were then incubated in a PCR thermocycler at 37° C. for 60 min (Cas12a cleavage) and at 65° C. for 15 min (Cas 12a inactivation). Following this, 1 μL of 2 μM probe T (0.4 μM) was added, and all six tubes were incubated at R.T for 1 h. Protein master mix and toehold switch were added to six paper discs like before, and then 0.5 μL of reaction solution from the PCR tubes was added to each of the paper discs. Absorbance readings were recorded over 2 h, and the images were captured post 2 h of protein synthesis.

Modularity of the Paper Sensor: 2.4 μM: 2.88 μM of Cas12a complex was prepared using crRNA_{Typhi} and added to 0.6 μM of probe AB. To this, 10 nM of ssDNA_{Typhi} (tDNA_{Typhi}), dsDNA_{Typhi} (tDNA_{Typhi}+CDNA_{Typhi}), SSDNAEnte (tDNA_{Ente}), and dsDNA_{Ente} (tDNA_{Ente}+CDNA_{Ente}) was then added separately and the reactions were incubated in a PCR thermocycler for the same cycle of Casl2a cleavage and inactivation. Next, 0.4 μM probe T was added to all, and the reaction was incubated at R. T for I h. Post incubation, 0.5 μL of the reaction was added to the protein master mix and toehold switch that was already on the paper. Absorbance and images were recorded the same way. The entire reaction was repeated using crRNA_{Ente} instead of crRNA_{Typhi} to demonstrate the modularity and programmability of assay.

Detection Sensitivity for dsDNA Targets: Cas12a reaction was set up using the same reaction components but with 0.05, 0.5, 5, 50, and 500 nM of dsDNA_{Typhi}. The addition of probeT and protein synthesis reaction remained unchanged.

Primer Screening for S. Typhimurium: For RPA, too, the reaction was prepared on ice. First, the pellet in eight tubes was rehydrated with 41.7 μL rehydration buffer. 2.4 μL of 10 μM 4F was added to tube 1-8, and 2.4 μL of 10 μM 1R, 2R, 3R, and 4R was added to tubes 1˜4 and 5-8, respectively. 1 μL of 1.28×10⁷ copies μL⁻¹ S. Typhimurium solution was added to tubes 1-4, and 1 μL RDI was added to tubes 5-8. Finally, 2.5 μL of 280 mM magnesium acetate (MgOAc) was added to start the RPA reaction. All tubes were incubated in a thermocycler at 37° C. for 30 min and at 65° C. for 15 min. 12 μL of all 8 tubes, 60 nM of Cas12a complex, and 60 nM of ssprobe (total volume=60 μL) were added to wells in a 96-well half area plate. The plate was then placed in a pre-heated plate reader (37° C.), and fluorescence was measured at, λexcitation of 485 nm and, λ_{excitation} of 520 nm for 60 min (FIG. 9).

Detection of S. Typhimurium Genome: RPA reaction was performed with 25, 100, 400, 1600, 6400, and 25600 copies of S. Typhimurium genome using 2F as forward primer and 4R as reverse primer. Cas12a reaction was performed with the same reagent concentrations, and 1 μL of amplicons was added as target. Post inactivation and addition of probe T, 0.5% μL of reaction was added to protein master mix and toehold switch. Protein expression was monitored for 2 h, after which the images were captured.

Real Sample Analysis of Salmonella: 273 g of green leaf lettuce leaves were washed and weighed out. These leaves were then blended with 200 mL of RDI in an Osterizer 12-speed blender. The blended solution was then passed through coffee filters to collect the lettuce juice in 50 mL tubes. 1.28×10⁷ copies μL⁻¹ S. Typhimurium solution was diluted to 400 copies μL⁻¹ using both lettuce juice and whole milk. The protocol for genomic DNA extraction followed was as provided by the kit manufacturer of Purelink Genomic DNA extraction kit. 100 μL of lettuce juice and milk containing 400 copies μL⁻¹ was used for extraction, ensuring 100 μL of RDI was used to elute the extracted DNA as well. 1 μL of the extracted genome from lettuce and milk solution was used as the template for the RPA reaction. Cas12a reaction and protein synthesis reaction had the same protocol as before.

Statistical Analysis: The experiments were performed in triplicate and the error bars were calculated by standard deviation (S.D.). Data are rep-resented as mean±S.D. Student's t-test was performed to calculate the P-values using graphpad software.

A such, conventional methods used to detect Salmonella in foods are laborious, time-consuming and permit detection of only higher levels of Salmonella. In the past, Yan et al. reported an electrochemical assay for sensing pathogenic nucleic acids that had an LOD of 1000 copies, while Zhang et al. recorded an LOD of 104 copies μL⁻¹. Furthermore, a previously published real-time PCR method for Salmonella could detect 100 copies of DNA with a threshold cycle (CT) value of 32.93. While various studies have explored the use of CRISPR-Cas12a for detecting pathogenic bacteria, our work stands out. We have developed a homogeneous, portable, paper-based assay that achieves not only higher sensitivity, but also shorter detection times compared to previous methods. Lastly, to showcase the capability of detecting the S. Typhimurium genome within a complex food matrix, we performed our assay using samples of green leaf lettuce and milk. To prepare our Salmonella-enriched samples, we added 400 copies of S. Typhimurium into both macerated green leaf lettuce and whole milk. Since most contaminated samples require extraction of genome from the bacteria, we carried out extractions on both our Salmonella-enriched and Salmonella-free samples.

The Salmonella-enriched lettuce sample resulted in a pronounced color change. The color transition with milk samples was not as pronounced, possibly due to higher protein and fat content in milk masking the reaction results on the paper. Despite the reduced color change for milk, the assay could successfully detect just 400 copies of genomic DNA in both samples on a small paper disc (FIG. 5B). Since Salmonella contaminations typically have much higher pathogen numbers to pose a serious health risk, our assay's high sensitivity is a remarkable achievement for preventing disease transmission. Furthermore, conventional methods of Salmonella detection, like other microbial assays, require a pre-enrichment step that can extend the detection process for days. Our protocol's exceptional detection capability negates the need for a pre-enrichment step and significantly reduces detection time for Salmonella.

We have successfully developed a synthetic biology cascade reaction on a paper substrate that allowed programming a toehold RNA switch for a genome of interest through CRISPR-Cas12a. This expanded versatility in detectable targets can be achieved without any need for structural changes to the toehold switch itself. Specifically, we demonstrated the detection of S. Typhimurium and S. Enteritidis targets using the same toehold switch simply by changing the crRNA responsible for recognition. Furthermore, by implementing an isothermal amplification reaction, we achieved visual detection of as little as 100 copies of the Salmonella genome on a small paper disc within a period of 4 h. Finally, to evaluate the practical application of this technology, we conducted an analysis on milk and lettuce samples, effectively demonstrating the utility of the paper sensor for investigating and controlling Salmonella infections. While the in vitro transcription and translation steps can be expensive, using 2 mm wide paper substrates for the diagnostic assay drastically reduces the cost. We believe this testing platform can be used for the detection of other pathogens as well.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of.” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

### REFERENCES

F. Medalla, W. Gu, C. R. Friedman, M. Judd, J. Folster, P. M. Griffin, R. M. Hoekstra, Emerging Infect. Dis. 2021, 27, 1662.; M. Majdinasab, A. Hayat, J. L. Marty, TrAC, Trends Anal. Chem. 2018, 107, 60.; R. R. A. Soares, R. G. Hjort, C. C. Pola, K. Parate, E. L. Reis, N. F. F. Soares, E. S. Mclamore, J. C. Claussen, C. L. Gomes, ACS Sens. 2020, 5, 1900.; Q. H. Nguyen, M. II Kim, Anal. Chem. 2020, 132, 116038. Y. Y. Broza, X. Zhou, M. Yuan, D. Qu, Y. Zheng, R. Vishinkin, M. Khatib, W. Wu, H. Haick, Chem. Rev. 2019, 119, 11761.; S. Slomovic, K. Pardee, J. J. Collins, Proc. Natl. Acad. Sci. USA 2015, 112, 14429.; A. A. Green, P. A. Silver, J. J. Collins, P. Yin, Cell 2014, 159, 925.; K. Pardee, A. A. Green, M. K. Takahashi, D. Braff, G. Lambert, J. W. Lee, T. Ferrante, D. Ma, N. Donghia, M. Fan, N. M. Daringer, I. Bosch, D. M. Dudley, D. H. O'Connor, L. Gehrke, J. J. Collins, Cell 2016, 165, 1255.; K. Pardee, A. A. Green, T. Ferrante, D. E. Cameron, A. DaleyKeyser, P. Yin, J. J. Collins, Cell 2014, 159, 940. M. K. Takahashi, X. Tan, A. J. Dy, D. Bra), R. T. Akana, Y. Furuta, N. Donghia, A. Ananthakrishnan, J. J. Collins, Nat. Commun. 2018, 9, 3347.; X. Tan, J. H. Letendre, J. J. Collins, W. W. Wong, Cell 2021, 184, 881.; F. Hong, D. Ma, K. Wu, L. A. Mina, R. C. Luiten, Y. Liu, H. Yan, A. A. Green, Cell 2020, 180, 1018.; J. S. Chen, E. Ma, L. B. Harrington, M. Da Costa, X. Tian, J. M. Palefsky, J. A. Doudna, Science 2018, 360, 436. C. W. Smith, N. Nandu, M. J. Kachwala, Y. S. Chen, T. B. Uyar, M. V. Yigit, Biochemistry 2020, 59, 1474.; C. W. Smith, M. J. Kachwala, N. Nandu, M. V. Yigit, ACS Synth. Biol. 2021, 10, 1785.; M. J. Kachwala, C. W. Smith, N. Nandu, M. V. Yigit, Anal. Chem. 2021, 93, 1934.; M. J. Kachwala, C. W. Smith, N. Nandu, M. V. Yigit, Nanoscale 2022, 14, 13500.; S. M. Z. Hossain, J. D. Brennan, Anal. Chem. 2011, 83, 8772.; S. C. Ricke, S. A. Kim, Z. Shi, S. H. Park, J. Appl. Microbiol. 2018, 125, 313.; C. Bayraç, F. Eyidog an, H. Avni Öktem, Biosens. Bioelectron. 2017, 98, 22.; R. Heymans, A. Vila, C. A. M. van Heerwaarden, C. C. C. Jansen, G. A. A. Castelijn, M. van der Voort, E. G. Biesta-Peters, PLOS One 2018, 13, 0206316.; L. Zhuang, J. Gong, Q. Li, C. Zhu, Y. Yu, X. Dou, X. Liu, B. Xu, C. Wang, Lett. Appl. Microbiol. 2014, 59, 658.; P. Kaiser, L. Rothwell, E. E. Galyov, P. A. Barrow, J. Burnside, P. Wigley, Microbiology 2000, 146, 3217.; R. Tarabees, M. S. A. Elsayed, R. Shawish, S. Basiouni, A. A. Shehata, J. Infect. Dev. Countries 2017, 11, 314.; Y. Shen, L. Xu, Y. Li, Compr. Rev. Food Sci. Food Saf. 2021, 20, 149.; Y. Yan, S. Ding, D. Zhao, R. Yuan, Y. Zhang, W. Cheng, Sci. Rep. 2016, 6, 18810.; X. Zhang, L. Guo, R. Ma, L. Cong, Z. Wu, Y. Wei, S. Xue, W. Zheng, S. Tang, J. Microbiol. Methods 2017, 139, 202.; K. N. Kasturi, T. Drgon, Appl. Environ. Microbiol. 2017, 83, 00644.; Q. Cai, H. Shi, M. Sun, N. Ma, R. Wang, W. Yang, Z. Qiao, J. Agric. Food Chem. 2022, 70, 16382.; L. Ma, L. Peng, L. Yin, G. Liu, S. Man, ACS Sens. 2021, 6, 2920.; B. Malorny, E. Paccassoni, P. Fach, C. Bunge, A. Martin, R. Helmuth, Appl. Environ. Microbiol. 2004, 70, 7046.; H. J. Cohen, S. M. Mechanda, W. Lin, Appl. Environ. Microbiol. 1996, 62, 4303.; S. A. Hice, K. D. Clark, J. L. Anderson, B. F. Brehm-Stecher, Anal. Chem. 2019, 91, 1113.; J. Ronholm, N. Nasheri, N. Petronella, F. Pagotto, Clin. Microbiol. Rev. 2019, 29, 837.; T. Inns, P. M. Ashton, S. Herrera-Leon, J. Lighthill, S. Foulkes, T., Jombart, Y. Rehman, A. Fox, T. Dallman, E. De Pinna, L. Browning, J. E. Coia, O. Edeghere, R. Vivancos, Epidemiol. Infect. 2017, 145, 289.; N. R. Thomson, D. J. Clayton, D. Windhorst, G. Vernikos, S. Davidson, C. Churcher, M. A. Quail, M. Stevens, M. A. Jones, M. Watson, A. Barron, A. Layton, D. Pickard, R. A. Kingsley, A. Bignell, L. Clark, B. Harris, D. Ormond, Z. Abdellah, K. Brooks, I. Cherevach, T. Chillingworth, J. Woodward, H. Norberczak, A. Lord, C. Arrowsmith, K. Jagels, S. Moule, K. Mungall, M. Sanders, Genome Res. 2008, 18, 1509.

What we claim is:

1. A method for visually detecting a Salmonella genome on a paper substrate, said method comprising: a) applying a sample that might contain the genome to the paper substrate; b) performing a cascade reaction including a CRISPR-Cas12a reaction utilizing a CRISPR-Cas12a-crRNA complex on the paper substrate; c) conducting a toehold RNA cascade reaction on the paper substrate; and d) visually detecting the presence of Salmonella genome based on the results of steps a), b) and c).

2. The method of claim 1, wherein the CRISPR-Cas12a reaction comprises: a) amplifying target sequences from the Salmonella genome when present in the sample using recombinase polymerase amplification (RPA) with a plurality of specific primers to create a plurality of amplicons; and b) activating the Cas12a-crRNA complex with the amplicons, wherein the crRNA is designed to complementary bind to the amplicons and activate Cas12a-crRNA complex.

3. The method of claim 1, wherein the toehold RNA cascade reaction comprises: a) degrading a DNA probe (probe AB) by the activated Cas12a-crRNA complex that is designed to complementary bind to a RNA probe (probe T); b) binding of the probe T designed to complementary bind to a toehold switch in an absence of the degraded probe AB; and c) activating the toehold switch, resulting in translation of a downstream LacZ gene and β-galactosidase synthesis.

4. The method of claim 3, wherein the β-galactosidase synthesis provides hydrolyzing a chromogenic substrate Chlorophenol Red-β-D-galactopyranoside (CPRG) resulting in a visible color change.

5. The method of claim 1, wherein the Salmonella genome is detected from about 100 copies or more of the genome from a Salmonella pathogen.

6. The method of claim 1, wherein the crRNA is specific for detecting S. Enteritidis (crRNA_{Ente}) or S. Typhi (crRNA_{Typhi}).

7. The method of claim 6, wherein the method is capable of separately detecting S. Typhimurium and S. Enteritidis targets.

8. A paper-based diagnostic device for visually detecting Salmonella genome, comprising: a) a paper substrate containing freeze-dried components for a cascade reaction including CRISPR-Cas12a reaction and toehold RNA cascade reaction; b) a Cas12a-crRNA complex wherein the crRNA is designed complementary to bind Salmonella genome amplicons; c) a toehold switch linked to a LacZ gene; and d) a chromogenic substrate for β-galactosidase.

9. The device of claim 8, wherein the freeze-dried components include at least one recombinase polymerase amplification (RPA) reagents, a Cas12a protein, a crRNA, a probe AB, a probe T, and at least one component for cell-free protein synthesis.

10. The device of claim 8, wherein the chromogenic substrate is Chlorophenol Red-β-D-galactopyranoside (CPRG).

11. A kit for visually detecting Salmonella genome, comprising: a) the paper-based diagnostic device of claim 8; b) instructions for use; and c) positive and negative control samples.

12. The kit of claim 11, further comprising specific primers for amplifying Salmonella genome targets.

13. The kit of claim 11, wherein the kit is capable of detecting multiple Salmonella serotype, including S. Enteritidis and S. Typhi.

14. A method for rapid field-deployable detection of Salmonella pathogens, comprising: a) applying a sample suspected of containing Salmonella to the paper-based diagnostic device of claim 8; b) incubating the device at a temperature suitable for RPA, CRISPR-Cas12a reaction, and toehold RNA cascade reaction; and c) visually observing a color change indicating the presence of Salmonella genome.

15. The method of claim 14, wherein the incubation temperature is between 37° C. and 42° C., and the incubation time is less than 2 hours.

16. A method for visually detecting a genome comprising: a) applying a sample that might contain the genome to a paper substrate; b) performing a cascade reaction including a CRISPR-Cas12a reaction on the paper substrate; c) conducting a toehold RNA cascade reaction on the paper substrate; and d) visually detecting the presence of a genome based on the results of steps a), b), and c).

17. The method of claim 16, wherein the CRISPR-Cas12a reaction comprises: a) amplifying a target sequence of the genome when present in the sample using recombinase polymerase amplification (RPA) with specific primers to create amplicons; b) activating a Cas12a-crRNA complex with said amplicons, wherein the crRNA is specific to the amplicons; c) degrading a DNA probe AB complementary to a RNA probe T by the activated Cas12a-crRNA complex; d) binding RNA probe T in the absence of DNA probe AB to a toehold switch and activating translation of a LacZ gene; e) synthesizing β-galactosidase; and f) hydrolyzing a chromogenic substrate by β-galactosidase.

18. The method of claim 17, wherein the chromogenic substrate is Chlorophenol Red-β-D-galactopyranoside (CPRG).

19. The method of claim 16, wherein the visual detection is performed by observing a color change on the paper substrate.

20. The method of claim 16, wherein the genome is a pathogen genome in the sample.


SEQUENCE LISTING (16 sequences)

SEQ ID NO: 1 - RNA, 36 nt, synthetic construct
  source 1..36: mol_type = other RNA
gacacaggacatgaaactgatgagaatagagcgaaa

SEQ ID NO: 2 - DNA, 36 bp, synthetic construct
  source 1..36: mol_type = other DNA
tttcgctctattctcatcagtttcatgtcctgtgtc

SEQ ID NO: 3 - DNA, 30 bp, synthetic construct
  source 1..30: mol_type = other DNA
ctcttcggcacaagtaatatcaacggtacg

SEQ ID NO: 4 - DNA, 32 bp, synthetic construct
  source 1..32: mol_type = other DNA
ctccgcaagttgagctttttccagatcttcac

SEQ ID NO: 5 - DNA, 32 bp, synthetic construct
  source 1..32: mol_type = other DNA
cacaagtaatatcaacggtacggtctctgtag

SEQ ID NO: 6 - DNA, 30 bp, synthetic construct
  source 1..30: mol_type = other DNA
ctccgcaagttgagctttttccagatcttc

SEQ ID NO: 7 - DNA, 33 bp, synthetic construct
  source 1..33: mol_type = other DNA
tcagtattgaggaaaaagaagggtcgtcgttag

SEQ ID NO: 8 - DNA, 33 bp, synthetic construct
  source 1..33: mol_type = other DNA
cagtattgaggaaaaagaagggtcgtcgttagg

SEQ ID NO: 9 - DNA, 31 bp, synthetic construct
  source 1..31: mol_type = other DNA
gagcagccgctcagtattgaggaaaaagaag

SEQ ID NO: 10 - DNA, 32 bp, synthetic construct
  source 1..32: mol_type = other DNA
cagtattgaggaaaaagaagggtcgtcgttag

SEQ ID NO: 11 - RNA, 41 nt, synthetic construct
  source 1..41: mol_type = other RNA
taatttctactaagtgtagattcgagatcgccaatcagtcc

SEQ ID NO: 12 - RNA, 41 nt, synthetic construct
  source 1..41: mol_type = other RNA
taatttctactaagtgtagatcgaataacatcctcaacttc

SEQ ID NO: 13 - DNA, 50 bp, synthetic construct
  source 1..50: mol_type = other DNA
tcgtcgttaggactgattggcgatctcgataaagtctctacagagaccgt

SEQ ID NO: 14 - DNA, 50 bp, synthetic construct
  source 1..50: mol_type = other DNA
acggtctctgtagagactttatcgagatcgccaatcagtcctaacgacga

SEQ ID NO: 15 - DNA, 50 bp, synthetic construct
  source 1..50: mol_type = other DNA
ggtatctgctgaagttgaggatgttattcgcaaagggatccgtcagacct

SEQ ID NO: 16 - DNA, 50 bp, synthetic construct
  source 1..50: mol_type = other DNA
aggtctgacggatccctttgcgaataacatcctcaacttcagcagatacc